(19) United States
(12) Patent Application Publication
(10) Pub. No.: US 2023/0181164 A1
(43) Pub. Date: Jun. 15, 2023

(54) ULTRASOUND OBSERVATION SYSTEM, OPERATION METHOD OF ULTRASOUND IMAGING APPARATUS, AND COMPUTER-READABLE RECORDING MEDIUM

(71) Applicant: OLYMPUS CORPORATION, Tokyo (JP)

(72) Inventor: Tomonao KAWASHIMA, Tokyo (JP)

(73) Assignee: OLYMPUS CORPORATION, Tokyo (JP)

(21) Appl. No.: 18/107,117

(22) Filed: Feb. 8, 2023

Related U.S. Application Data
(63) Continuation of application No. PCT/JP2020/034779, filed on Sep. 14, 2020.

(51) Int. Cl. A61B 8/08

(52) U.S. Cl. CPC A61B 8/5207

(57) ABSTRACT

An ultrasound observation system includes a processor includes hardware. The processor is configured to: receive an echo signal based on ultrasound scanning of a scan region of a subject; set first regions in the scan region, each one of the first regions including second regions; calculate frequency spectra in the respective second regions based on an analysis of the echo signal; calculate a plurality of pieces of feature data based on the frequency spectra; calculate a statistical value of the plurality of pieces of feature data in the first regions; set filters for the respective first regions based on the statistical value; perform a filtering process with the filters on the echo signal to calculate a second echo signal; and generate ultrasound image data based on an amplitude of the second echo signal, frequency curves of the filters differing from each other depending on the statistical value.

### CROSS-REFERENCE TO RELATED APPLICATION

This application is a continuation of International Application No. PCT/JP2020/034779, filed on Sep. 14, 2020, the entire contents of which are incorporated herein by reference.

### BACKGROUND

### 1. Technical Field

The present disclosure relates to an ultrasound observation system that observes a subject using ultrasound, an operation method of an ultrasound imaging apparatus, and a computer-readable recording medium.

### 2. Related Art

In the present application, the term “subject” is used as a generic term for a living body or a dead body of a human or an animal, or an organ or an organ derived therefrom. These are all made up of tissues. An ultrasound imaging apparatus that observes a subject using ultrasound waves is widely known. The ultrasound imaging apparatus transmits an ultrasound wave to a subject and performs a predetermined signal process on an ultrasound echo backscattered by the subject, thereby acquiring information on the subject. Among these ultrasound imaging apparatuses, for example, an apparatus that generates a B-mode image expressing the intensity of an ultrasound echo based on the information is known. On the other hand, there is also known an ultrasound imaging apparatus that analyzes the frequency of backscattered ultrasound echoes to generate a tissue characterization image representing features of tissue characterization in a subject (see, for example, JP 2006-524115 A and WO 2012/063930 A). The tissue characterization image can represent features of the scattering body that are less than or equal to the resolution of the B-mode image.

Among them, the device disclosed in WO 2012/063930 A can display the B-mode image and the tissue characterization image described above side by side on a display screen. An operator such as a doctor observes a B-mode image and a tissue characterization image disposed on a screen and perform, a diagnosis.

### SUMMARY

In some embodiments, an ultrasound observation system includes a processor includes hardware. The processor is configured to: receive an echo signal based on ultrasound scanning of a scan region of a subject; set first regions in the scan region, each one of the first regions including second regions; calculate frequency spectra, in the respective second regions based on an analysis of the echo signal; calculate a plurality of pieces of feature data based on the frequency spectra; calculate a statistical value of the plurality of pieces of feature data in the first regions; set filters for the respective first regions based on the statistical value; perform a filtering process with the filters on the echo signal to calculate a second echo signal; and generate ultrasound image data based on an amplitude of the second echo signal, frequency curves of the filters differing from each other depending on the statistical value.

In some embodiments, provided is an operation method of an ultrasound imaging apparatus. The method includes: receiving an echo signal based on ultrasound scanning of a scan region of a subject; setting first regions in the scan region, each one of the first regions including second regions; calculating frequency spectra in the respective second regions based on an analysis of the echo signal; calculating a plurality of pieces of feature data based on the frequency spectra; calculating a statistical value of the plurality of pieces of feature data in the first regions; setting filters for the respective first regions based on the statistical value; performing a filtering process with the filters on the echo signal to calculate a second echo signal; and generating ultrasound image data based on an amplitude of the second echo signal, frequency curves of the filters differing from each other depending on the statistical value.

In some embodiments, provided is a non-transitory computer-readable recording medium with an executable program stored thereon. The program causes an ultrasound imaging apparatus to execute: receiving an echo signal based on ultrasound scanning of a scan region of a subject; setting first regions in the scan region, each one of the first regions including second regions; calculating frequency spectra in the respective second regions based on an analysis of the echo signal; calculating a plurality of pieces of feature data based on the frequency spectra; calculating a statistical value of the plurality of pieces of feature data in the first regions; setting filters for the respective first regions based on the statistical value; performing a filtering process with the filters on the echo signal to calculate a second echo signal; and generating ultrasound image data based on an amplitude of the second echo signal, frequency curves of the filters differing from each other depending on the statistical value.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram for explaining scattering of ultrasonic waves for a tissue having a relatively large scattering body size;

FIG. 2 is a diagram illustrating the frequency spectra of an ultrasonic wave transmitted to the tissue illustrated in FIG. 1 and an ultrasonic wave returned by backscattering;

FIG. 3 is a diagram for explaining scattering of ultrasonic waves for a tissue having a relatively small scattering body size;

FIG. 4 is a diagram illustrating the frequency spectra of an ultrasonic wave transmitted to the tissue illustrated in FIG. 3 and an ultrasonic wave returned by backscattering;

FIG. 5 is a diagram for explaining ultrasound scanning by the ultrasound transducer;

FIG. 6 is a view illustrating an example of a size of a tissue in part of a scanning range of ultrasound scanning using the ultrasound transducer illustrated in FIG. 5;

FIG. 7 is a diagram illustrating an example of the frequency spectra of a transmission wave at the time of ultrasound scanning;

FIG. 8 is a diagram illustrating an example of the frequency spectra of a reception wave at the time of ultrasound scanning;

FIG. 9 is a block diagram illustrating a configuration of an ultrasound observation system including an ultrasound imaging apparatus according to an embodiment of the disclosure;

FIG. 10 is a flowchart illustrating an outline of processing executed by the ultrasound imaging apparatus according to the embodiment of the disclosure;

FIG. 11 is a flowchart illustrating a flow of processing of ultrasound scanning illustrated in FIG. 10;

FIG. 12 is a diagram for explaining sound rays generated by ultrasound scanning;

FIG. 13 is a flowchart illustrating a flow of a feature data map generation process illustrated in FIG. 10;

FIG. 14 is a diagram for describing calculation of frequency feature data using the frequency spectra;

FIG. 15 is a diagram for describing an example of a feature data map;

FIG. 16 is a diagram for explaining calculation of a variation grade;

FIG. 17 is a diagram for explaining identification of a variation grade;

FIG. 18 is a diagram for explaining an example of a variation map;

FIG. 19 is a diagram for explaining a relationship between a variation grade and a filter coefficient;

FIG. 20 is a diagram illustrating an example of a relationship between a frequency and an input/output intensity ratio in a variation grade;

FIG. 21 is a flowchart illustrating a flow of a B-mode image data generation process illustrated in FIG. 10;

FIG. 22 is a diagram illustrating a configuration of a filter unit illustrated in FIG. 9;

FIG. 23 is a diagram for explaining B-mode image data;

FIG. 24 is a flowchart illustrating a flow of a display image data generation process illustrated in FIG. 10;

FIG. 25 is a diagram (part 1) illustrating an example of a display mode of a B-mode image on a display screen;

FIG. 26 is a diagram (part 2) illustrating an example of a display mode of a B-mode image on a display screen;

FIG. 27 is a diagram for explaining identification of a variation grade in the first modification;

FIG. 28 is a diagram for explaining identification of a variation grade in the second modification;

FIG. 29 is a diagram for explaining a relationship between a variation grade and a filter coefficient in the second modification;

FIG. 30 is a diagram (part 1) for explaining the aspect of the ultrasound transducer;

FIG. 31 is a diagram (part 2) for explaining the aspect of the ultrasound transducer; and

FIG. 32 is a diagram for explaining a feature data image generated based on feature data.

### DETAILED DESCRIPTION

Hereinafter, modes for carrying out the disclosure (hereinafter, referred to as “embodiments”) will be described with reference to the accompanying drawings.

### Embodiments

### I. Principle Relationship Between Scattering Body And Spectrum of Reception Wave

### I-i. General Principle

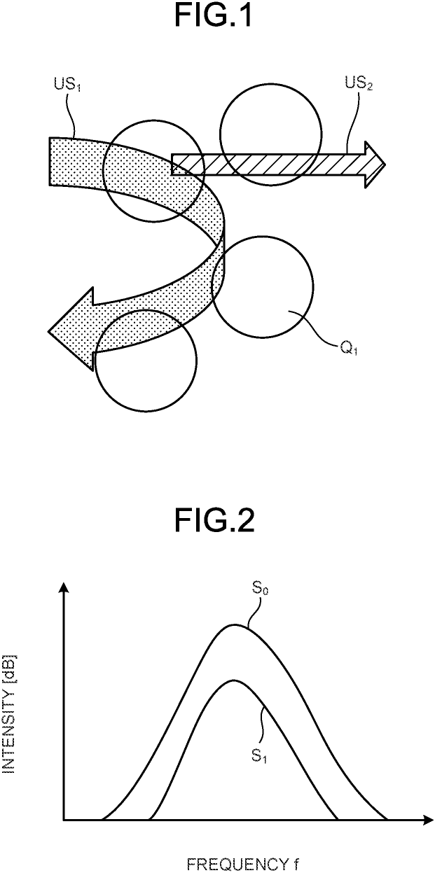

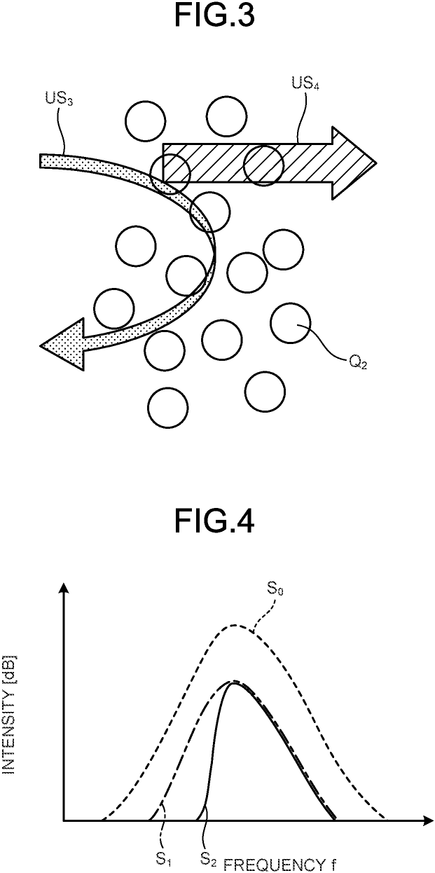

FIG. 1 is a diagram illustrating scattering of ultrasonic waves for a tissue having a relatively large scattering body size. FIG. 2 is a diagram illustrating the frequency spectra of an ultrasonic wave (hereinafter, also simply referred to as a “transmission wave”) transmitted to the tissue illustrated in FIG. 1 and an ultrasonic wave (hereinafter, also simply referred to as a “reception wave”) returned by backscattering. FIG. 3 is a diagram illustrating scattering of ultrasonic waves in a tissue having a relatively small scattering body size. FIG. 4 is a diagram illustrating frequency spectra of a transmission wave and a reception wave to and from the tissue illustrated in FIG. 3. The frequency spectrum of the reception wave illustrated in FIGS. 2 and 4 is actually observed as a frequency distribution of intensity and a voltage amplitude of an echo signal obtained by performing acousto-electrical conversion on the reception wave. The tissue scattering body Q₁ illustrated in FIG. 1 is larger than the tissue scattering body Q₂ illustrated in FIG. 3.

In general, the frequency spectrum of the reception wave tends to vary depending on the properties of the tissue of the subject scanned with the ultrasonic wave. This is because the frequency spectra is affected by the size, number density, acoustic impedance, and the like of the scattering body that scatters the ultrasonic wave. The frequency spectra is particularly susceptible to the size of the scattering body. The tissue characterization is, for example, a characteristic of a tissue such as a malignant tumor (cancer), a benign tumor, an endocrine tumor, a mucinous tumor, a normal tissue, a cyst, or a vessel when the subject is a human tissue.

Meanwhile, scatter in an ultrasonic wave refers to a phenomenon in which an ultrasonic wave hits an irregular boundary surface or a scattering body which is a microreflector and spreads in all directions. Furthermore, back scatter refers to a phenomenon in which scattering returns backward, that is, in the direction of the sound source. In general, a transmission wave to a tissue including a scattering body is less likely to be scattered as the transmission wave is long, compared with the size of the scattering body, and is more likely to be scattered as the transmission wave is short, compared with the size of the scattering body. In other words, the smaller the scattering body is compared with the wavelength of the transmission wave, the less likely the transmission wave is to be scattered, and the larger the scattering body is, the more likely the transmission wave is to be scattered. The same applies to backscattering.

Here, a case where the same transmission wave is incident on each tissue illustrated in FIGS. 1 and 3 and is backscattered, and then a reception wave is received is considered. In general, a transmission wave is not a single wavelength but is typically composed of many frequency components. In the scattering body Q₁ having a relatively large size, most of the frequency components of the transmission wave are backscattered back (see FIG. 1). At this time, the reception wave is reduced with respect to the transmission wave. The intensity of the frequency spectrum S₁ of the reception wave at each frequency is smaller than the intensity of the frequency spectrum S₀ of the transmission wave at each frequency over the entire frequency (see FIG. 2) .

On the other hand, in the scattering body Q₂ having a small size, a component having a lower frequency in the transmission wave passes through the scattering body Q₂ and hardly returns as a reception wave (see FIGS. 3 and 4). At this time, the reception wave is further reduced as scattering body compared with the reception wave of the Q₁. This is particularly noticeable at low frequencies. The intensity of the frequency spectrum S₂ of the reception wave is smaller than the intensity of the frequency spectrum S₀ of the transmission wave and smaller than the intensity of the frequency spectra S₁ over the entire frequency (see FIG. 4).

As can be seen from the above description, the lower the frequency, the more clearly the difference in the size of the scattering body appears in the reception wave. The present application focuses on this point of the general principle. Note that, in this discussion, attenuation between the transmission point (sound source) and the tissue and between the tissue and the reception point is not considered. In a case where there is attenuation, compensation according to the distance between the transmission and reception point (sound source) and the tissue is required after reception.

### I-ii. When Tissue Is Scanned With Same Ultrasound Probe

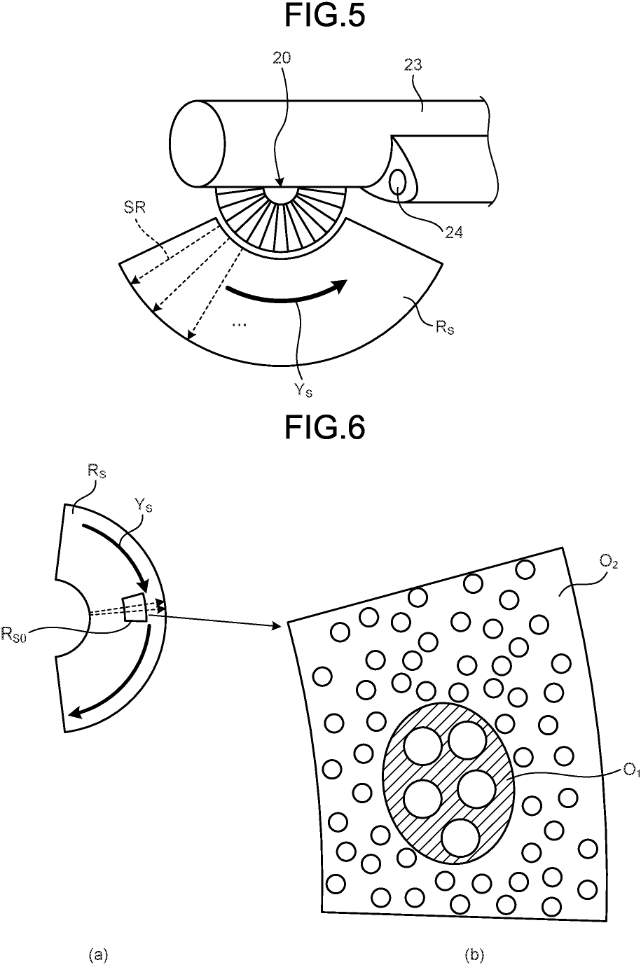

FIG. 5 is a diagram for explaining ultrasound scanning by the ultrasound transducer. Hereinafter, an example of scanning a subject using the convex type ultrasound transducer 20 illustrated in FIG. 5 will be described. The ultrasound transducer 20 transmits an ultrasound beam (transmission wave), and receives an ultrasound wave (reception wave) that has been backscattered by a scattering body included in the tissue in the subject and returned. FIG. 5 illustrates this transmission/reception direction as SR. The entire fan-shaped scanning range Rₛ is scanned with the ultrasonic wave by repeating transmission and reception while moving the transmission and reception direction of the ultrasonic wave in the scanning direction Yₛ in the plane (scanning face) using the ultrasound transducer 20.

FIG. 6 is a diagram illustrating an example of a size of a tissue in part of a scanning range of ultrasound scanning using the ultrasound transducer 20 illustrated in FIG. 5. (a) of FIG. 6 illustrates the scanning range Rₛ, and (b) of FIG. 6 illustrates an example of the tissue corresponding to a partial region Rₛ₀ of the scanning range Rₛ. For example, it is assumed that there are tissues O₁ and O₂ in which the sizes of the scattering bodies are different from each other at a position corresponding to the region Rₛ₀ (see (b) of FIG. 6).

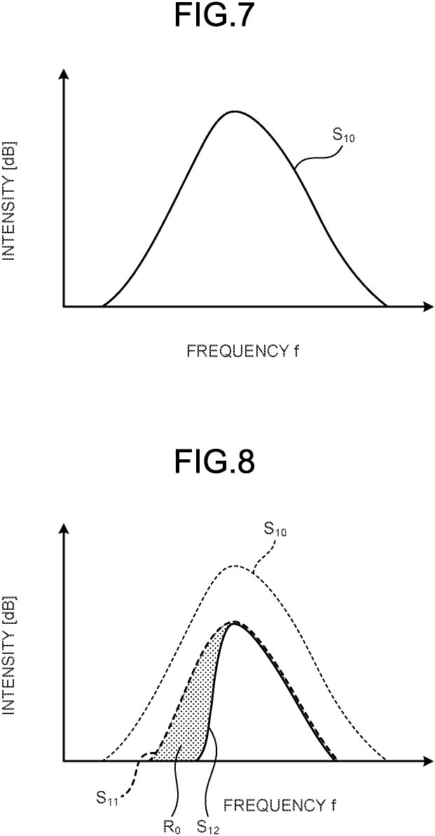

FIG. 7 is a diagram illustrating an example of the frequency spectra of the transmission wave at the time of ultrasound scanning. FIG. 8 is a diagram illustrating an example of the frequency spectra of the reception wave at the time of ultrasound scanning. FIG. 8 illustrates the frequency spectra in the region Rₛ₀ in (b) of FIG. 6. When the transmission wave of the frequency spectra S₁₀ illustrated in FIG. 7 is transmitted to the region Rₛ₀, the reception wave from the tissue O₁ indicates the frequency spectra S₁₁, and the reception wave from the tissue O₂ indicates the frequency spectra S₁₂. In FIG. 8, the frequency spectra S₁₀, S₁₁, and S₁₂ are indicated by a dotted line, a broken line, and a solid line, respectively. As can be seen from FIG. 8, the difference in spectrum intensity is large at the low frequency.

Here, the frequency feature data (hereinafter, it is also simply referred to as a “feature data”) is calculated by a slope or an intercept of a straight line approximated from the frequency spectra, and a combination thereof. The above-described difference in the spectra between the tissues (corresponding to the region R₀) appears as the difference in the frequency feature data. It is a principle of the present application to utilize this difference. Hereinafter, the configuration, operation, and effect of the device for guiding and utilizing the difference will be described.

### II. Configuration of Present Embodiment

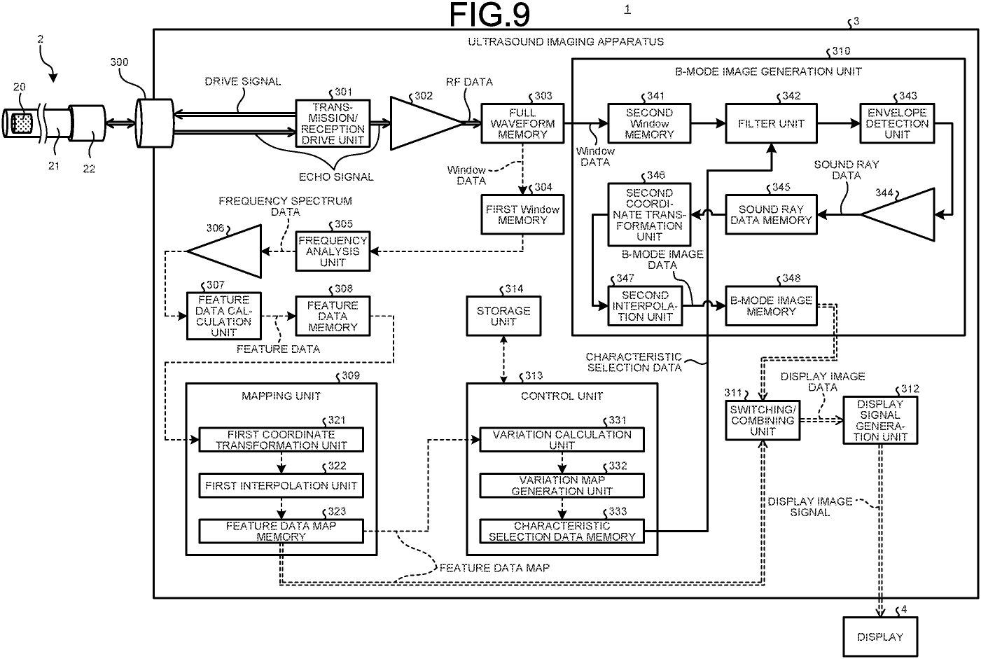

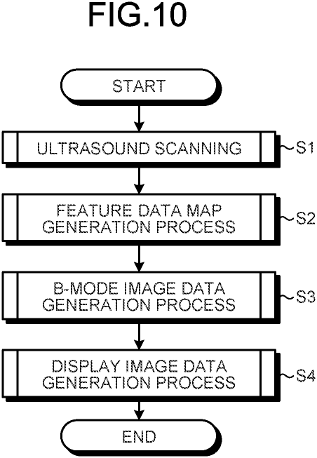

FIG. 9 is a block diagram illustrating a configuration of an ultrasound observation system 1 including an ultrasound imaging apparatus 3 according to an embodiment of the disclosure. The ultrasound observation system 1 illustrated in the figure includes an ultrasound probe 2 that transmits an ultrasound wave to a subject and receives the ultrasound wave backscattered by the subject, an ultrasound imaging apparatus 3 that generates an ultrasound image based on an echo signal acquired by the connected ultrasound probe 2, and a display 4 that displays the ultrasound image generated by the ultrasound imaging apparatus 3. In the block diagram, double line arrows indicate transmission of electric signals and data related to ultrasound scanning and discretization, solid line arrows indicate transmission of electric signals and data related to B-mode image data generation, broken line arrows indicate transmission of electric signals and data related to feature data calculation, and double broken line arrows indicate transmission of electric signals and data related to image display.

An ultrasound endoscope will be described as an example of the ultrasound probe 2 of the present embodiment. The ultrasound probe 2 includes a long and flexible insertion unit 21 to be inserted into the subject, a connector 22 connected to the proximal end of the insertion unit 21, and a distal end unit 23 located at the distal end of the insertion unit 21. The distal end unit 23 has, for example, a configuration illustrated in FIG. 5. The distal end unit 23 includes a convex type ultrasound transducer 20 for scanning the subject with an ultrasound wave and an optical observation window 24 for optically observing the inside of the subject. The optical observation window 24 is connected to an imaging optical system such as an optical lens and an imaging element (not illustrated) provided inside the distal end unit 23 and the insertion unit 21. The ultrasound transducer 20 is an array including a large number of elements, and respective elements are connected to a transmission/reception drive unit 301 (described later) via the distal end unit 23, the insertion unit 21, the connector 22, and a connection unit 300 (described later) of the ultrasound imaging apparatus 3 by a signal line (not illustrated) .

The ultrasound imaging apparatus 3 includes the connection unit 300, the transmission/reception drive unit 301, an A/D converter 302, a full waveform memory 303, a first Window memory 304, a frequency analysis unit 305, a first log amplifier 306, a feature data calculation unit 307, a feature data memory 308, a mapping unit 309, a B-mode image generation unit 310, a switching/combining unit 311, a display signal generation unit 312, a control unit 313, and a storage unit 314. Details of the processing of respective units will be described later.

The connection unit 300 includes a plurality of connection pins connected to the plurality of respective signal lines and is fixed to the housing of the ultrasound imaging apparatus 3. The connector 22 is detachable from the connection unit 300. That is, the ultrasound probe 2 provided with the connector 22 is detachable from the ultrasound imaging apparatus 3, and can be connected to the connection unit 300 by replacing with another type of ultrasound probe. The connection unit 300 electrically connects the ultrasound probe 2 and the ultrasound imaging apparatus 3 via a signal line.

The mapping unit 309 includes a first coordinate transformation unit 321, a first interpolation unit 322, and a feature data map memory 323.

The control unit 313 includes a variation calculation unit 331, a variation map generation unit 332, and a characteristic selection data memory 333. The control unit 313 reads an operation program, calculation parameters of each process, data, and the like stored in the storage unit 314 from the storage unit, and controls the ultrasound imaging apparatus 3 in an integrated manner by causing respective units to execute various types of calculation processing related to an operation method. The control unit 313 has a function as an image generation control unit of the present application.

In addition, the B-mode image generation unit 310 includes a second Window memory 341, a filter unit 342, an envelope detection unit 343, a second log amplifier 344, a sound ray data memory 345, a second coordinate transformation unit 346, a second interpolation unit 347, and a B-mode image memory 348. The B-mode image generation unit 310 of the present embodiment corresponds to an image data generation unit of the present application. Note that the image data generation unit may include the switching/combining unit 311 and the display signal generation unit 312 in addition to the B-mode image generation unit 310.

The B-mode image generation unit 310, the frequency analysis unit 305, the feature data calculation unit 307, the mapping unit 309, the switching/combining unit 311, the display signal generation unit 312, and the control unit 313 described above are realized using a general-purpose processor such as a central processing unit (CPU) having calculation and control functions, a dedicated integrated circuit that executes a specific function such as an application specific integrated circuit (ASIC) or a field programmable gate array (FPGA), or the like. Note that a plurality of units including at least some of the above units may be configured using a common general-purpose processor, a dedicated integrated circuit, or the like. Furthermore, some circuits of the transmission/reception drive unit 301 can be realized by a dedicated integrated circuit.

In addition, the full waveform memory 303, the first Window memory 304, the feature data memory 308, the feature data map memory 323, the characteristic selection data memory 333, the second Window memory 341, the sound ray data memory 345, and the B-mode image memory 348 are configured using, for example, a hard disk drive (HDD), a synchronous dynamic random access memory (SDRAM), or the like.

Here, the ultrasound imaging apparatus 3 further includes the storage unit 314 that stores calculation parameters, data, and the like of each processing in addition to the above-described various memories. The storage unit 314 stores, for example, an operation program of the ultrasound imaging apparatus 3, data required for various types of processing, information required for logarithmic conversion processing (see the following Expression (1), for example, values of a and V_{c}), information about a window function (Hamming, Hanning, Blackman, etc.) required for frequency analysis processing, and the like. Furthermore, the storage unit 314 may store the generated B-mode image data, frequency spectrum data, and the like. The storage unit 314 is configured using, for example, an HDD, an SDRAM, or the like.

In addition, the storage unit 314 includes, as an additional memory, a non-transitory computer-readable recording medium in which an operation program for executing an operation method of the ultrasound imaging apparatus 3 is installed in advance, for example, a read only memory (ROM) (not illustrated). The operation program can be widely distributed by being recorded in a computer-readable recording medium such as a portable hard disk, a flash memory, a CD-ROM, a DVD-ROM, or a flexible disk. Note that the ultrasound imaging apparatus 3 can acquire the above-described operation program, various types of data, and various types of information by an input/output unit (not illustrated) connected to these recording media and record the acquired operation program, various types of data, and various types of information in the storage unit 314. Furthermore, the ultrasound imaging apparatus 3 can acquire the above-described operation program, various types of data, and various types of information by downloading the operation program, various types of data, and various types of information via a communication network by a communication circuit (not illustrated) and record the acquired operation program, various types of data, and various types of information in the storage unit 314. The communication network here is implemented by, for example, an existing public network, LAN, WAN, or the like, and may be wired or wireless.

### III. Action of Present Embodiment

### III-i. Overview

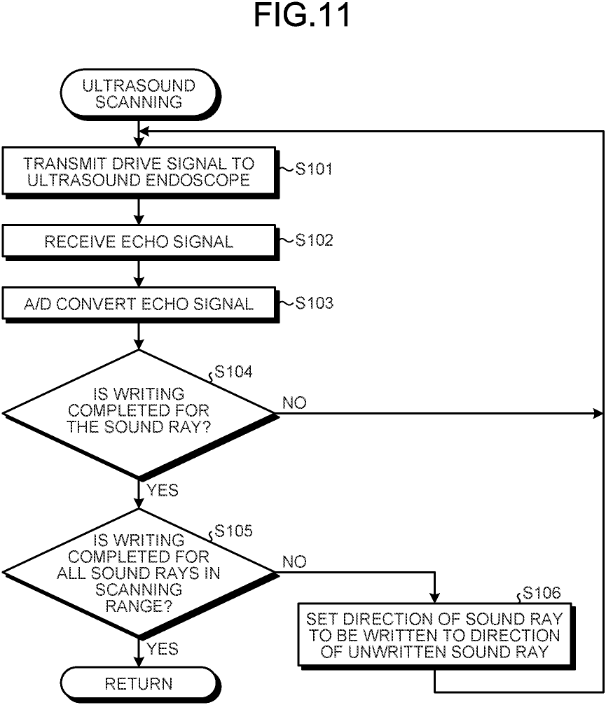

Next, processing executed by the ultrasound imaging apparatus 3 will be described. FIG. 10 is a flowchart illustrating an outline of processing executed by the ultrasound imaging apparatus. Under the control of the control unit 313, the ultrasound imaging apparatus 3 scans the subject with ultrasound waves, generates an ultrasound image based on the received echo signal, and displays the ultrasound image on the display 4.

The ultrasound imaging apparatus 3 first causes the ultrasound probe 2 to perform ultrasound scanning (step S1). Thereafter, the ultrasound imaging apparatus 3 generates the feature data map based on the echo signal received from the ultrasound probe 2 (step S2). The ultrasound imaging apparatus 3 generates B-mode image data based on the generated feature data map (step S3), and displays a B-mode image based on the generated B-mode image data on the display 4 (step S4) .

### III-ii. Step S1 Ultrasound Scanning, Discretization

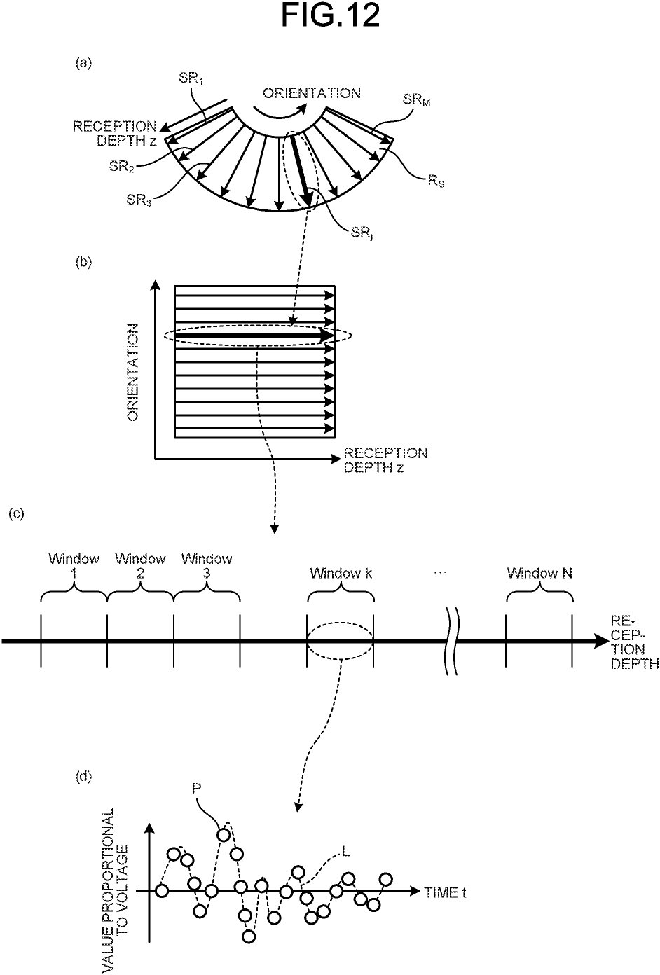

First, a flow of processing of ultrasound scanning and discretization in step S1 illustrated in FIG. 10 will be described. Step S1 corresponds to the flow of the double line arrows in FIG. 9. Hereinafter, a flow of processing of ultrasound scanning and discretization will be described with reference to FIG. 11.

In step S101, the transmission/reception drive unit 301 transmits a drive signal to the ultrasound transducer 20 based on a control signal from the control unit 313. The ultrasound transducer 20 transmits a transmission wave based on the drive signal to the subject.

Specifically, the transmission/reception drive unit 301 applies a different delay to a drive signal composed of a high-voltage pulse having a predetermined waveform to output the drive signal to each signal line connected to the ultrasound transducer 20 at a predetermined transmission timing. The predetermined waveform, the delay, and the predetermined transmission timing are based on the control signal from the control unit 313. The drive signal is transmitted to the ultrasound transducer 20 via each pin and each signal line in the connection unit 300 of the ultrasound imaging apparatus 3, and the connector 22, the insertion unit 21, and the distal end unit 23 of the ultrasound probe 2. The ultrasound transducer 20 converts the drive signal into an ultrasound pulse that is a transmission wave and emits the ultrasound pulse in a specific direction of the subject. This transmission direction is determined by the value of the delay applied to the drive signal to respective elements.

In step S102, the transmission/reception drive unit 301 receives an echo signal based on the ultrasound echo returned from the scattering body received by the ultrasound transducer 20. Specifically, the transmission wave is backscattered by the scattering body included in the tissue existing in the irradiation direction (hereinafter, it is also simply referred to as a “sound ray”) in the subject, and an ultrasound echo is generated. Then, the ultrasound echo is received as a reception wave by the ultrasound transducer 20. The ultrasound transducer 20 converts the reception wave into an electrical echo signal expressed by a voltage change to output the electrical echo signal to each signal line. The transmission/reception drive unit 301 receives the echo signal via each signal line and each pin in the distal end unit 23, the insertion unit 21, and the connector 22 of the ultrasound probe 2, and the connection unit 300 of the ultrasound imaging apparatus 3. The echo signal received here is an electrical radio frequency (RF) signal.

In step S103, the A/D converter 302 performs an A/D conversion process on the echo signal received by the transmission/reception drive unit 301 to generate digital data (hereinafter, referred to as RF data). Specifically, the A/D converter 302 first amplifies the received echo signal. The A/D converter 302 performs processing such as filtering on the amplified echo signal, and then performs sampling at an appropriate sampling frequency (for example, 50 MHz) and discretization (so-called A/D conversion processing). In this way, the A/D converter 302 generates discretized RF data from the amplified echo signal. The A/D converter 302 writes the RF data to the full waveform memory 303.

Note that the frequency band of the drive signal transmitted by the transmission/reception drive unit 301 is set to a wide band that substantially covers the linear response frequency band of the ultrasound transducer 20 when the ultrasound transducer 20 performs electroacoustic conversion on the drive signal into an ultrasound pulse (transmission wave). Furthermore, the various processing frequency band of the echo signal in the A/D converter 302 is set to a wide band that substantially covers the linear response frequency band of the ultrasound transducer when the ultrasound transducer performs the acousto-electrical conversion on the ultrasound echo (reception wave) into the echo signal. As a result, it is possible to prevent, as much as possible, a problem that a so-called effective band included in both the linear response frequency band of the electroacoustic conversion and the linear response frequency band of the acousto-electrical conversion in the ultrasound transducer 20 is impaired by the action of the transmission/reception drive unit 301 and the A/D converter 302. As a result, the frequency spectra approximation processing described later can be executed in a wide band as much as possible, and accurate approximation can be performed.

In step S104, the control unit 313 determines whether writing of the RF data to the full waveform memory 303 has been completed for the sound ray. When determining that the writing is not completed (step S104: No), the control unit 313 returns to step S101 and repeats the above-described processing for the unwritten RF data. On the other hand, when determining that writing has been completed for the sound ray (step S104: Yes), the control unit 313 proceeds to step S105.

In step S105, the control unit 313 determines whether writing has been completed for all the sound rays within the scanning range. When determining that writing of all the sound rays is not completed (step S105: No), the control unit 313 proceeds to step S106.

In step S106, the control unit 313 changes the value of the delay to set the direction of the sound ray to be written to the direction of the sound ray that has not yet been written. After setting the direction of the sound ray, the control unit 313 returns to step S101 and causes each unit to repeat the above-described processing for an unwritten sound ray.

On the other hand, when determining that writing has been completed for the sound ray (step S105: Yes), the control unit 313 ends the ultrasound scanning process.

As described above, by repeating steps S101 to S105 while changing the delay of the element, the ultrasound transducer 20 scans the fan-shaped scanning range Rₛ while moving the transmission direction of the ultrasound in the scanning direction Yₛ of FIG. 5, and writes the RF data of all the sound rays in the scanning range Rₛ to the full waveform memory 303.

Here, the relationship between the scanning and the data in the full waveform memory 303 will be specifically described with reference to FIG. 12. FIG. 12 is a diagram for explaining sound rays generated by ultrasound scanning. (a) of FIG. 12 is a diagram schematically illustrating a scanning range and a sound ray of the ultrasound transducer. (b) of FIG. 12 illustrates that the depth and orientation of respective sound rays are aligned in two orthogonal directions. A scanning range illustrated in (a) of FIG. 12 has a fan shape. Note that, in (a) of FIG. 12, a path (sound ray) along which the ultrasonic wave reciprocates is represented by a straight arrow. In (a) of FIG. 12, for convenience of subsequent description, each sound ray is numbered as 1, 2, 3, ... in order from the start of scanning, and a first sound ray is defined as SR₁, a second sound ray is defined as SR₂, a third sound ray is defined as SR₃, ..., a j-th sound ray is defined as SRⱼ, ..., and an M-th (last) sound ray is defined as SR_{M}. In addition, in (a) and (b) of the reception depth FIG. 12, reception depth of the RF data on each sound ray is described as z. In a case where the ultrasonic pulse emitted from the surface of the ultrasound transducer is backscattered in the tissue at the reception depth z and returns to the ultrasound transducer as an ultrasound echo, there is a relationship of z = D/2 between the round-trip distance D and the reception depth z.

(c) of FIG. 12 is a diagram schematically illustrating a data array in the RF data corresponding to the sound ray SRⱼ. Window 1, Window 2, Window 3, ..., Window k, ..., and Window N are sections obtained by dividing the sound ray SRⱼ at predetermined depth intervals. A set of RF data in each Window i s defined c. s Window data. Each Window da t a includes RF data of a plurality of sample points. In the RF data on the sound ray SRⱼ, the RF data and the Window data located on the right side represent the RF data and the Window data from a deeper position when measurement is made along the sound ray SRⱼ from the ultrasound transducer. Each Window corresponds to the second region of the present application in the set of RF data. Note that the reception depth is associated with the reception time of the ultrasound echo.

(d) of FIG. 12 is a diagram for describing data of each sample point in Window k. The vertical axis of the graph illustrated in (d) of FIG. 12 indicates a value corresponding to the displacement of the ultrasound echo at the time when the ultrasound echo is received and proportional to the voltage. Further, as described above, the RF data P on the sound ray SRⱼ is RF data which is sampled from the echo signal by the A/D conversion process in the A/D converter and discretized. Note that a broken line L illustrated in (d) of FIG. 12 indicates a waveform of an original ec h o signal in Window k.

### III-iii. Step S2 Feature Data Map Generation Process

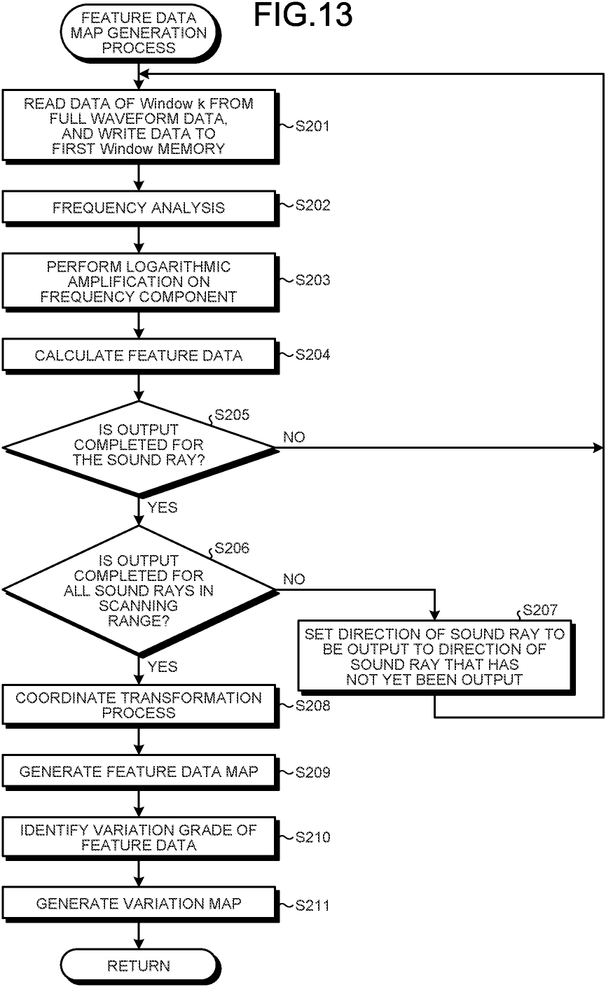

Next, the feature data map generation process in step S2 illustrated in FIG. 10 will be described. Step S2 corresponds to a flow of the broken line arrow in FIG. 9. Hereinafter, the flow of the feature data map generation process in step S2 will be described with reference to FIG. 13.

In step S201, the control unit 313 reads the Window data stored in the full waveform memory 303. Specifically, the control unit 313 reads Window data of a k-th Window (Window k) on the j-th sound ray SRj stored, in the full waveform memory 303. Although step S201 is repeated in a loop of FIG. 13 as described later, the control unit 313 sets the initial value of j to 1 and the initial value of k to 1 in advance. Therefore, at the first time of the loop, the control unit 313 reads the data of Window 1. Then, the control unit 313 writes the read Window data to the first Window memory 304.

In step S202, the frequency analysis unit 305 performs frequency analysis on the Window data. Specifically, the frequency analysis unit 305 performs a fast Fourier transform (FFT) , which is a type of frequency analysis, on Window data of Window k stored in the first Window memory 304 to calculate data (hereinafter, referred to as “frequency spectrum data”) of the frequency spectrum in Window k. Here, the frequency spectrum data represents a “frequency distribution of intensity and voltage amplitude of the echo signal obtained from the reception depth z (that is, a certain reciprocating distance D) at Which Window of Processing Target Exists”.

In the present embodiment, a case where a frequency distribution of a voltage amplitude of the echo signal is used as the frequency spectrum will be described. A case where the frequency analysis unit 305 generates data of the frequency spectrum based on the frequency component V (f) of the voltage amplitude will be described as an example, f represents a frequency. The frequency analysis unit 305 _{J} the frequency component V (f) of the amplitude divides the frequency component V (f) of the amplitude (practically, the voltage amplitude of_ the echo signal) of the RF data by the reference voltage V_{c}, performs logarithmic conversion processing of taking- the common logarithm (log) of it and expressing the common logarithm in decibels, and then multiplies the common logarithm, by an appropriate positive constant a to generate frequency spectrum data S(f) , of the subject, given by the following Expression (1). Note that the constant α is, for example, 20.

$$\text{S} \left(\text{f}\right) = α ⋅ log \left\{\text{V} \left(\text{f}\right) / {\text{V}}_{\text{c}}\right\}$$

The frequency analysis unit 305 outputs the frequency spectrum data S(f) to the first log amplifier 306. The data output to the first log amplifier 306 is data in which values each proportional to a digit in which the amplitude or the intensity of the echo signal indicating the intensity of backscattering of the ultrasonic pulse is expressed in 10 digits are disposed along the transmission/reception direction (depth direction) of the ultrasonic pulse, as shown in Formula (1) .

In step S203, the first log amplifier 306 performs logarithmic amplification on each frequency component of the input frequency spectrum data to output the amplified frequency spectrum data.

In step S204, the feature data calculation unit 307 approximates the frequency spectrum data after logarithmic amplification output from the first log amplifier 306 with a straight line, and calculates the feature data of the frequency spectrum data using the straight line. The feature data calculation unit 307 outputs the calculated feature data to the feature data memory 308.

The calculation of the feature data by the feature data calculation unit 307 will be specifically described with reference to FIG. 14. For example, the feature data calculation unit 307 performs a single regression analysis in the frequency band U to obtain the regression line Lₛ of the frequency spectrum data Sₛ. At this time, the feature data calculation unit 307 calculates the slope a₁ and the intercept b₁ of the acquired regression line L_{S} as the feature data. Then, the mid-band fit c₁ = a₁f_{M} + b₁, which is a value on a regression line at the center frequency (that is, a “midband”) f_{M} = (f_{L} + f_{H}) /2 of the frequency band U, is also calculated as the feature data. The frequency spectrum data S_{S} is approximated to a linear expression by expressing the frequency spectrum data S_{S} with parameters (slope a₁, intercept b₁, midband fit c₁), of the linear expression that characterizes the regression line L_{S}.

The feature data calculation unit 307 outputs, to the feature data memory 308, the value of the type that is set to output as the feature data among the slope a₁, the intercept b₁, and the midband fit c₁.

Among the three pieces of feature data calculated from the data of the frequency spectra, the slope a₁ and the intercept b₁ are considered to have a correlation with the size of the scattering body that scatters the ultrasonic wave, the scattering intensity of the scattering body, the number density (concentration) of the scattering body, and the like. The midband fit c₁ provides the voltage amplitude and the intensity of the echo signal at the center within the effective frequency band. Therefore, it is considered that the mid-band fit c₁ has a certain degree of correlation with the luminance of the B-mode image in addition to the size of the scattering body, the scattering intensity of the scattering body, and the number density of the scattering body.

In step S205, the control unit 313 determines whether the output of the feature data has been completed for the sound ray whose feature data is to be calculated. Specifically, when k = N (the number of the last Window on the sound ray SRⱼ), the control unit 313 determines that the output of the feature data has been completed for all the Windows on the sound ray SRⱼ, and when k < N, it determines that the output is not completed. Thereafter, in a case where the control 313 that the unit determines output of the feature data is not completed for all the Windows (step S205: No), 1 is added to the value of k, the process returns to step S201, and the above-described process is repeated for the Window data of Window k (the value of k is the same as k + 1 before the addition). In this way, the process moves to a window whose feature data is not output. On the other hand, when the control unit 313 determines that the output of the feature data is completed (step S205: Yes), the process proceeds to step S206.

In step S206, the control unit 313 determines whether the output of the feature data has been completed for all the sound rays within the scanning range R_{S}. Specifically, when j = M (the number of the last sound ray in the scanning range R_{S}), the control unit 313 determines that the output of the feature data has been completed for all the sound rays in the scanning range R_{S}, and when j < M, it determines that the output of the feature data is not completed. Thereafter, when the control unit 313 determines that the output of the feature data is not completed for all the sound rays (step S206: No), the process proceeds to step S207.

In step S207, the control unit 313 sets the direction of the sound ray to be output as the direction of the sound ray that has not yet been output. Specifically, the control unit 313 adds 1 to the value of j, returns to step S201, and repeats the above-described processing for the sound ray of the sound ray SRⱼ (the value of j is the same as j + 1 before addition). In this manner, the process proceeds to a sound ray whose feature data is not yet output.

On the other hand, when the control unit 313 determines that the output of the feature data has been completed for all the sound rays (step S206: Yes), the process proceeds to step S208.

In step S208, the first coordinate transformation unit 321 of the mapping unit 309 allocates the feature data stored in the feature data memory 308 in correspondence with each pixel position of the image in the B-mode image data. In the present embodiment, for convenience of description, each pixel will be described as being disposed on orthogonal coordinates.

In step S209, the first interpolation unit 322 interpolates the feature data at the position where the feature data does not exist in the above-described orthogonal coordinates. The first interpolation unit 322 calculates the feature data at the position at which the feature data is to be interpolated using the feature data around the position at which the feature data is to be interpolated. As the surrounding feature data used for interpolation, for example, the feature data at a position adjacent to the position at which the feature data is to be interpolated in the vertical direction and the horizontal direction and the feature data at a position in contact with the position at which the feature data is to be interpolated in the oblique direction are used. The first interpolation unit 322 writes all the pieces of feature data including the interpolated feature data to the feature data map memory 323. In steps S208 and 3209 described above, the mapping unit 309 generates the feature data map and stores the feature data map in the feature data map memory 323. The mapping unit 309 outputs the feature data map stored in the feature data map memory 323 to the switching/combining unit 311 and the control unit 313.

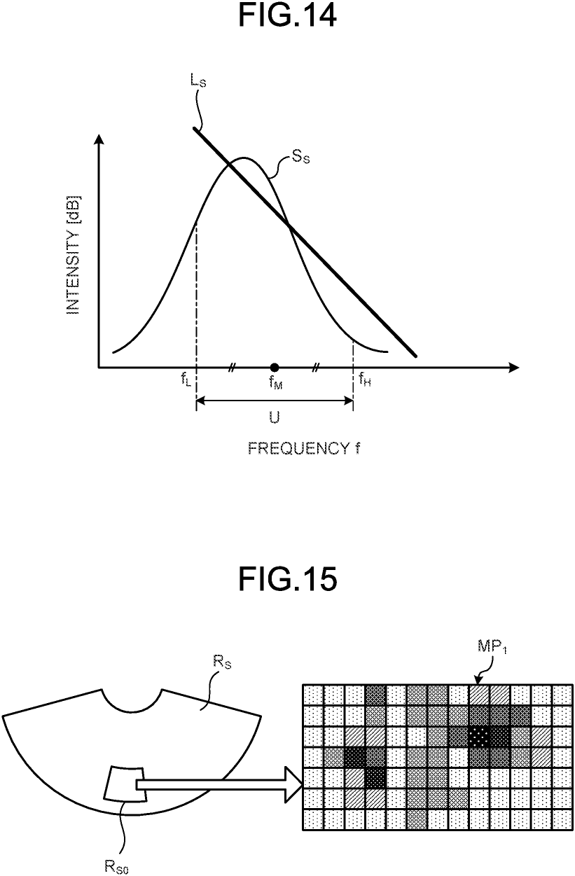

FIG. 15 is a diagram illustrating an example of a feature data map. FIG. 15 illustrates an example of a feature data map corresponding to a region R_{S0} of part of the scanning range R_{S}. The scanning range R_{S} and the region R_{S0} are described above in the description of FIG. 5. Note that a feature data map MP₁ illustrated in FIG. 15 is represented by a rectangle for descrition. In the feature data map MP₁, the feature data is expressed in units of Window data, and in FIG. 15, the lower the feature data, the thinner the hatching, and the higher the feature data, the darker the hatching.

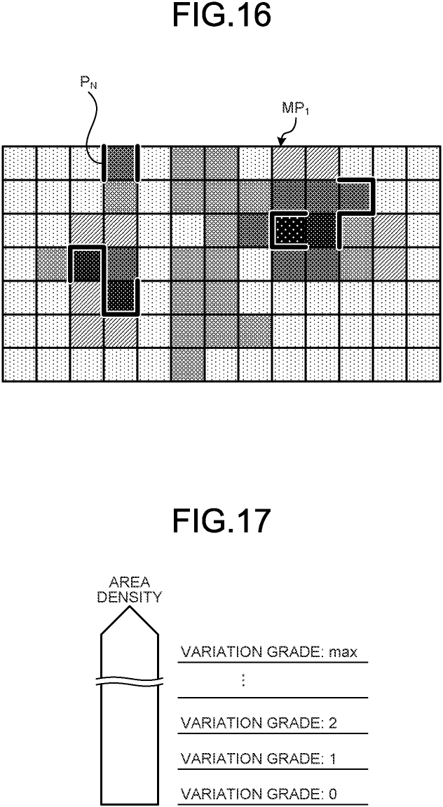

In step S210, the control unit 313 identifies a variation grade based on the feature data map. Specifically, the variation calculation unit 331 first reads the feature data map from the feature data map memory 323, and extracts an adjacent place of a window where a difference in the feature data at an adjacent position is equal to or larger than a threshold value. FIG. 16 is a diagram for explaining calculation of a variation grade. The variation calculation unit 331 extracts an adjacent place P_{N} where the difference in the feature data is equal to or larger than a threshold value. In FIG. 16, the extracted adjacent places are indicated by thick lines. The threshold value used at this time corresponds to the first threshold value of the present application.

Thereafter, the variation calculation unit 331 counts the number of the extracted adjacent places for each of the divided regions (see FIG. 18) . As an example, in FIG. 16, there are 15 extracted adjacent places. Here, the divided region is a region obtained by dividing the scanning range R_{S}, and is a region including a plurality of windows. As the divided region, a region in which the scanning range is divided by the sound ray direction and a curve (iso-depth line) connecting the same depth will be described as an example, but the dividing method is not limited thereto. The divided region corresponds to the first regions of the present application. The region R_{S0} is also one of these divided regions. The variation calculation unit 331 divides the counted number by the actual area of the divided region to calculate the number density of the divided region of the number of Window adjacent places having a difference in the feature data equal to or greater than the threshold value. Here, the area density is calculated as the number density.

The variation calculation unit 331 reads, from the storage unit 314, an association table in which the area density and the variation grade are associated with each other, where the association table is stored in the storage unit 314 in advance. Then, the variation calculation unit 331 refers to the association table and identifies a variation grade corresponding to the area density for each divided region.

FIG. 17 is a diagram for explaining identification of a variation grade. In the variation grade, the numerical value of the grade increases as the area density increases. That is, when the variation in the feature data in the divided region is large, the variation grade of the divided region is also large.

In step S211, the variation map generation unit 332 associates the position and size of the divided region with the variation grade, generates a variation map, and outputs the variation map to a characteristic selection data memory. In addition, the variation map generation unit 332 also outputs, to the characteristic selection data memory, a relationship table in which a variation grade is associated with information on a filter coefficient of the filter unit 342 to be described later. Specifically, the variation map generation unit 332 first associates the position and size of the divided region with the variation grade. The variation map generation unit 332 generates a variation map by this association.

FIG. 18 is a diagram for explaining an example of the variation map. In the variation map, a variation grade is set for each divided region T_{R}. The variation map generation unit 332 outputs the generated variation map to the characteristic selection data memory 333.

Thereafter, the variation map generation unit 332 reads, from the storage unit 314, a relationship table in which the variation grade is associated with the information on the filter coefficient stored in advance in the storage unit 314. Then, the variation map generation unit 332 outputs this relationship table to the characteristic selection data memory 333. In this manner, the characteristic selection data memory 333 stores two tables of the “variation map” and the “relationship table in which the variation grade is associated with the filter coefficient information”. Hereinafter, these two are referred to as characteristic selection data.

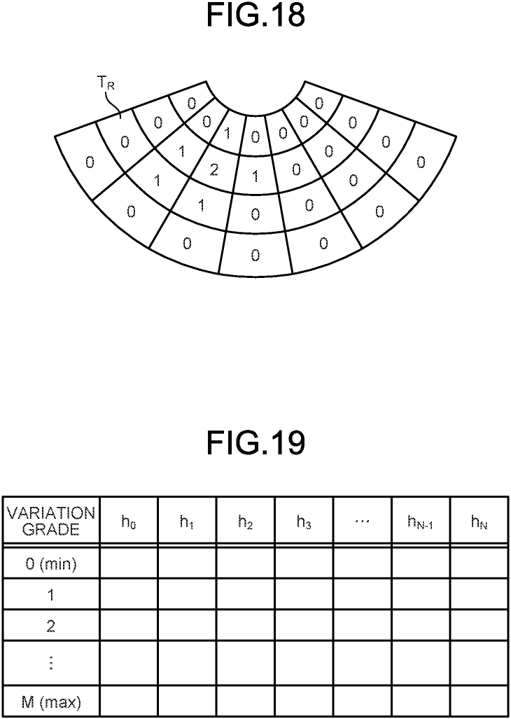

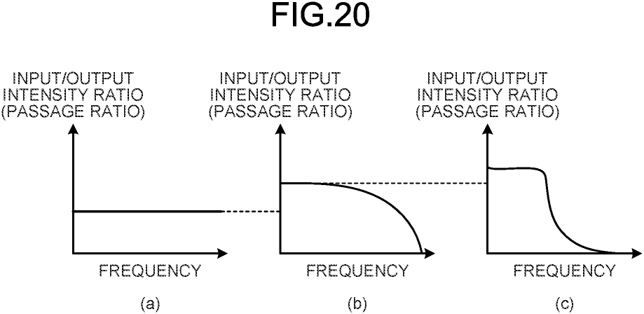

FIG. 19 is a diagram for describing a relationship table in which the variation grade is associated with information about a filter coefficient. The filter coefficient corresponds to an input/output intensity ratio of each frequency component in a case where a plurality of parameters of h₀, h_{1,} h_{2,} ..., h_{N-1,} and h_{N} is set as one set, and the signal passing through the filter unit 342 to be described later is discretely broken down in a plurality of frequencies. However, the filter coefficient is not the input/output intensity ratio itself. The filter coefficient does not directly correspond to the input/output intensity ratio. The input/output intensity ratio corresponds to the passage ratio of the signal in the 342. In FIG. 19, a set of filter coefficients filter unit 342. In FIG. 19, a set of filter coefficients h₀, h₁, h₂, ..., h_{N-1}, h_{N} is set corresponding to each of variation grades 0, 1, 2, 3, ..., M.

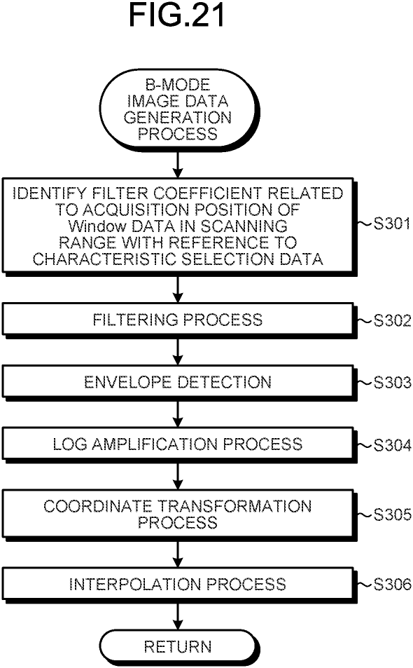

FIG. 20 is a diagram illustrating an example of a relationship between a frequency and an input/output intensity ratio in the filter unit 342 of the B-mode image generation unit 310 described later. In FIG. 20, the relationship is illustrated for each variation grade. (a) of FIG. 20 illustrates an example of the input/output intensity ratio with respect to the frequency corresponding to the filter coefficient in a case where the variation grade is 0. (b) of FIG. 20 illustrates an example of the input/output intensity ratio with respect to the frequency corresponding to the filter coefficient in a case where the variation grade is 1. (c) of FIG. 20 illustrates an example of the input/output intensity ratio with respect to the frequency corresponding to the filter coefficient in a case where the variation grade is 2. In the present embodiment, when the variation grade increases, the input/output intensity ratio at the low frequency increases, and further, the input/output intensity ratio at the high frequency decreases. Note that the filter coefficient with the variation grade of 0 in (a) of FIG. 20 is a coefficient in which any frequency is not enhanced.

### III-iv. Step S3 B-Mode Image Data Generation Process

Next, the B-mode image data generation process in step S3 illustrated in FIG. 10 will be described. Step S3 corresponds to the flow of the solid line arrow in FIG. 9. Hereinafter, the flow of the B-mode image data generation process in step S3 will be described with reference to FIG. 21.

In step S301, the filter coefficient related to the acquisition position of the Window data in the scanning range is identified with reference to the characteristic selection data. Specifically, first, the control unit 313 outputs the position information about the window to be processed in the scanning range Rₛ to the B-mode image generation unit 310. The B-mode image generation unit 310 reads the corresponding Window data from the full waveform memory 303 based on the position information, and writes the read Window data to the second Window memory 341. The filter unit 342 reads the Window data stored in the second Window memory 341. The filter unit 342 reads the characteristic selection data (variation map and relationship table in which the variation grade is associated with filter coefficient information illustrated in FIG. 19) from the characteristic selection data memory 333. Then, the filter unit 342 identifies the filter coefficients h₀, h₁, h₂, ..., h_{N+1}, and h_{N} related to the acquisition position of the Window data read from the second Window memory from the position information about the Window by referring to the variation map and the “relationship table in which the variation grade is associated with the information about the filter coefficient”.

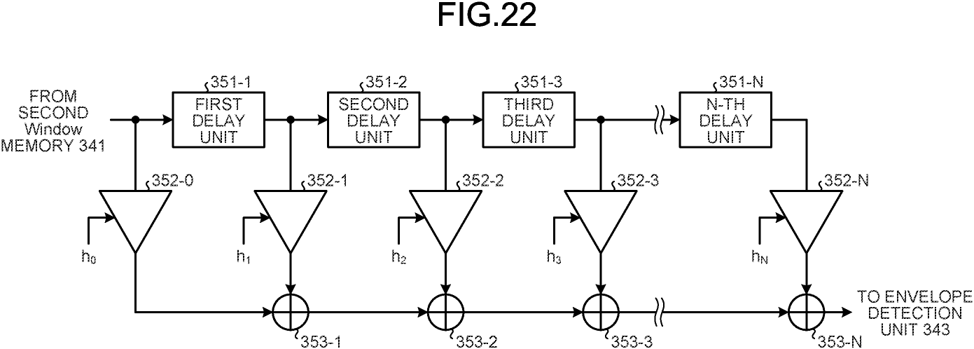

In step S302, the filter unit 342 performs a filtering process of the Window data using the identified filter coefficient. FIG. 22 is a diagram illustrating a configuration of the filter unit illustrated in FIG. 9. The filter unit 342 includes a first delay unit 351-1, a second delay unit 351-2, a third delay unit 351-3, ..., and an N-th delay unit 351-N, a 0-th amplification unit 352-0, a first amplification unit 352-1, a second amplification unit 352-2, a third amplification unit 352-3,..., and an N-th amplification unit 352-N, and a first addition unit 353-1, a second addition unit 353-2, a third addition unit 353-3, ..., and an N-th addition unit 353-N. Each delay unit outputs the Window data to each amplification unit at the same predetermined delay time. In addition, Window data and a filter coefficient corresponding to a variation grade are input to each amplification unit. For example, the window data is input from the second Window memory 341 to the 0-th amplification unit 352 0 without delay, and the filter coefficient h₀ is input thereto. The 0-th amplification unit 352 0 multiplies the Window data by the filter coefficient h₀ to output the result to the first addition unit 353-1. In addition, the Window data delayed by a predetermined delay time from the first delay unit 351-1 is input to the first amplification unit 352-1, and the filter coefficient h₁ is input thereto. The first amplification unit 352-1 multiplies the Window data by the filter coefficient h₁ to output the result to the first addition unit 353-1. The first addition unit 353-1 adds the Window data input from the 0-th amplification unit 352 0 and the first amplification unit 352-1, to output the result to the second addition unit 353-2.

As described above, the filter unit 342 delays the Window data according to the delay time, multiplies the Window data according to the filter coefficient, adds the Window data to the cumulative addition result of the Window data so far to output the window data after addition to the addition unit in the subsequent stage. When all the values of the filter coefficients h₀, h₁, h₂, ..., h_{N+1}, and h_{N}, are determined, the input/output intensity ratio (passage ratio) of each frequency component is uniquely determined. As described above, the frequency curve of the input/output intensity ratio (passage ratio) of the filter unit 342 changes as follows according to the variation grade of the position within the scanning range Rₛ of the Window data.

When the variation grade is 0, the frequency curve is the curve illustrated in (a) of FIG. 20, and any frequency components are not enhanced.

When the variation grade is 1, the frequency curve is the curve illustrated in (b) of FIG. 20, and the low frequency component is enhanced and the high frequency component is suppressed.

When the variation grade is 2, the frequency curve is the curve illustrated in (c) of FIG. 20, and the low frequency component is further enhanced and the high frequency component is further suppressed.

As described above, the filter unit 342 enhances the low frequency component of the Window data according to the variation grade of the feature data of the divided region to which the Window belongs by the filtering process, suppresses the high frequency component, and outputs the result to the envelope detection unit 343.

In step S303, the envelope detection unit 343 performs envelope detection on the Window data output from the filter unit 342. Specifically, the envelope detection unit 343 performs band pass filtering and envelope detection on the Window data, and generates digital sound ray data representing the amplitude or intensity of the echo signal.

In step S304, as in the first log, amplifier 306, the second log amplifier 344 performs logarithmic amplification on the input sound ray data (corresponding to the voltage amplitude of the echo signal) to output the sound ray data after logarithmic amplification (corresponding to the voltage amplitude after logarithmic amplification). The second log amplifier 344 outputs the amplified sound ray data to the sound ray data memory 345.

S305, the second coordinate transformation In step S305, unit 346 acquires the sound ray data stored in the sound ray data memory 345, and performs coordinate transformation such that the sound ray data can spatially correctly represent the scanning range. In this manner, the second coordinate transformation unit 346 rearranges the sound ray data.

In step S306, the second interpolation unit 347 performs interpolation processing between the sound ray data to fill a gap between the sound ray data to generate B-mode image data. The B-mode image is a gray scale image in which values of red (R), green (G), and blue (B), which are variables in a case where the RGB color system is used as the color space, are matched. The second interpolation unit 347 outputs the generated B-mode image data to the B-mode image memory 348. Note that the second interpolation unit 347 may perform a signal process on the sound ray data using a known technique such as gain processing or contrast processing.

FIG. 23 is a diagram for explaining B-mode image data. The B-mode image G_{B} is an image on which the filtering process is performed according to the variation grade set for each divided region. In FIG. 23, the larger the variation grade is, the darker the color is hatched. The B-mode image G_{B} is an image in which the low frequency component is enhanced as the hatched region is darker.

As described above, the control unit 313 causes the B-mode image generation unit 310 to generate B-mode image data obtained by performing the process on a plurality of divided regions included in the scanning range of the ultrasound scanning according to the feature data corresponding to the divided region. Here, the “plurality of divided regions included in the scanning range of the ultrasound scanning” refers to a region obtained by dividing an image (for example, a B-mode image) in which the scanning range is visualized based on an echo signal obtained by the ultrasound scanning.

### III-v. Step S4 Display Image Data Generation Process

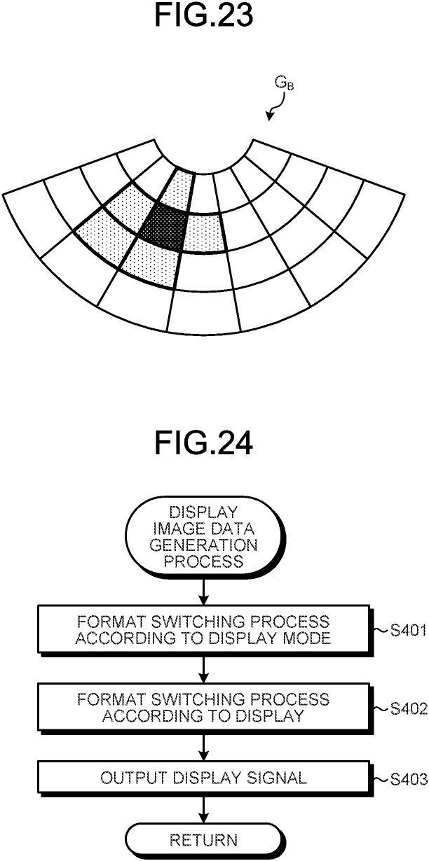

Next, the display image data generation process in step S4 illustrated in FIG. 10 will be described. Step S4 corresponds to the flow of the double broken line arrow in FIG. 9. The flow of step S4 will be described below with reference to FIG. 24.

In step S401, the switching/combining unit 311 executes a process of switching to a display format corresponding to the set display mode. Specifically, first, the switching/combining unit 311 reads the feature data map stored in the feature data map memory 323 and the B-mode image data stored in the B-mode image memory 348. Thereafter, the switching/combining unit 311 performs a format process corresponding to either single display in which only the B-mode image is displayed or parallel display in which the B-mode image and the feature data map are displayed side by side according to the set display mode. Only necessary image data may be read according to the display mode.

In step S402, the display signal generation unit 312 performs a format process according to the display format of the display 4 that displays an image. The type of the display format of the display 4 includes a monitor size, resolution, and the like. The display signal generation unit 312 generates a display signal to be displayed on the display 4, for example, by performing a predetermined process such as thinning of data according to a display range of an image in the display 4 or gradation processing.

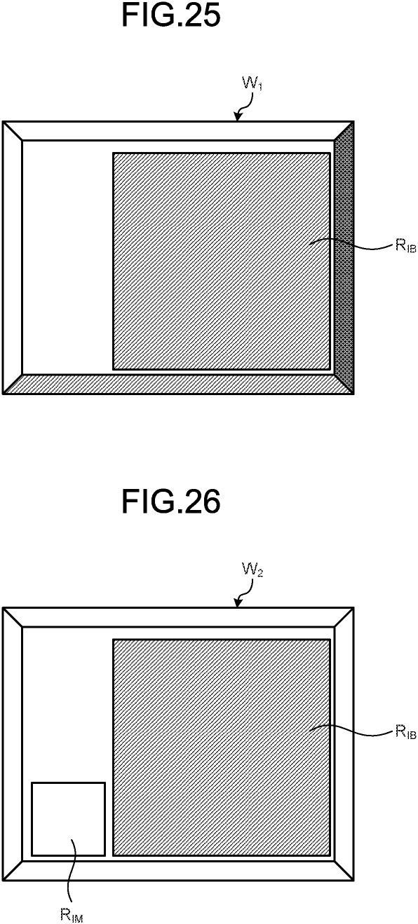

In step S403, the control unit 313 issues a command to the display signal generation unit 312, causes the display 4 to output the display signal generated by the display signal generation unit 312, and causes the display 4 to display an image. FIGS. 25 and 26 are diagrams illustrating an example of a display mode of the B-mode image on the display screen. FIG. 25 is a diagram illustrating a case where the B-mode image alone is displayed. FIG. 26 is a diagram illustrating a case where the B-mode image and the feature data map are displayed in parallel. When the B-mode image alone is displayed, the B-mode image G_{B} illustrated in FIG. 23 is displayed on the display screen W₁ of the display 4, for example, in the B-mode image display area R_{IB}. In addition, when the B-mode image and the feature data map are displayed in parallel, the B-mode image G_{B} illustrated in FIG. 23 is displayed on thedisplay screen W₂ of display4, example, B-mode image display area R_{IB}, and the feature data map MP₁ (however, instead of the orthogonal coordinates, the feature data map in which the coordinate system matches that of the B-mode image) illustrated in FIG. 15 or the variation map illustrated in FIG. 18 is displayed in the feature data map display area R_{IM}.

Each display screen may further display information necessary for observation and diagnosis.

### IV. Effects of Present Embodiment

In the embodiment described above, the variation grade is calculated based on the difference between the feature calculated based on the difference between the data of adjacent Windows in the feature data map, and the filter coefficient of the filtering process executed by the filter unit 342 is identified according to the variation grade. As described above, a difference in the size of the scattering body between the tissues largely appears in the feature data. By setting the filter coefficient at the time of generating the B-mode image data using the characteristics of the feature data, the B-mode image in which the specific frequency is enhanced is generated in the region (divided region in the embodiment) in which the variation in feature data is large.

In general, normal tissues are often uniform tissues composed of scattering bodies each having a uniform size. On the other hand, abnormal tissues such as tumors exhibit various tissues, and a plurality of types of tissues is often mixed. In this plurality of types of tissues, sizes of scattering bodies of the respective tissues are different from each other, for example, as in O₁ and O₂ of the divided region R_{SO} in (b) of FIG. 6. Therefore, the divided region included in the abnormal tissue has a larger variation in the feature data than the divided region included in the normal tissue. In the divided region including the abnormal tissue, by setting the filter coefficient of the filtering process executed when the B- mode image data is generated based on the feature data and the variation grade thereof, for example, the low frequency component sensitive to the difference in the size of the scattering body is enhanced, and the abnormal tissue is easily distinguished from the normal tissue by visual recognition. As a result, when this ultrasound image is used, it is easy to search for a lesion having characteristics in tissue characterization. On the other hand, since this ultrasound image is generated based on the B-mode image, this can be achieved without lowering the resolution. Therefore, according to the present embodiment, it is possible to display an image in which it is easy to search for a lesion having characteristics in tissue characterization without impairing spatial resolution.

### First Modification

Next, the first modification will be described. FIG. 27 is a diagram for describing identification of a variation grade in the first modification. The ultrasound observation system according to the first modification has the same configuration as the ultrasound observation system of the above-described embodiment. The first modification is different from the above-described embodiment in the processing content of the variation calculation unit 331.

The variation calculation unit 331 reads the feature data map MP₂ from the feature data map memory 323, and extracts a Window in which the feature data of each Window data is equal to or more than a threshold value. In FIG. 27, the extracted Window is surrounded by a thick line frame P_{N1} . The threshold value used at this time corresponds to the second threshold value of the present application.

Thereafter, the variation calculation unit 331 counts the number of extracted Windows for each divided region. The variation calculation unit 331 divides the counted number by the actual area of the divided region to calculate the number density of the divided region of the number of Windows in which the value of the feature data is equal to or greater than the threshold value. Here, the area density is calculated as the number density.

The subsequent processing is similar to that of the above-described embodiment.

In the first modification described above, the variation grade is calculated based on the area density of the Window in which the value of the feature data is equal to or larger than the threshold value in the feature data map, and the filter coefficient of the filtering process executed by the filter unit 342 is identified according to the variation grade. Therefore, in the first modification, as in the embodiment, it is easy to confirm the notable position of the tissue characterization in the ultrasound image having higher spatial resolution than the image based on the feature data. As a result, it is possible to display an image in which it is easy to search for a lesion having characteristics in tissue characterization without impairing spatial resolution.

Note that, in the present modification, a semi-bounded section defined by the value of the feature data being “greater than or equal to a threshold value” is used. However, depending on the type of the feature data, a semi-bounded section defined by the value of the feature data being “less than or equal to a threshold value” may be used. Furthermore, a bounded section defined by the value of the feature data being from a certain threshold value or more to a certain threshold value or less may be used. This is because depending on the type of the feature data, there are various cases such as a case where the feature data monotonically increases with respect to the size of the scattering body, a case where the feature data monotonically decreases, and a case where the feature data does not monotonically increase or decrease. Therefore, in order to easily confirm the notable position of the tissue characterization, it i ; desirable to set the section of the feature data for counting the Window before calculating the area density to the section in which the difference of the abnormal tissue with respect to the normal tissue appears.

### Second Modification

Next, the second modification will be described. The ultrasound observation system according to the second modification has the same configuration as the ultrasound observation system of the above-described embodiment. The second modification is different from the embodiment in the processing content of the variation calculation unit 331.

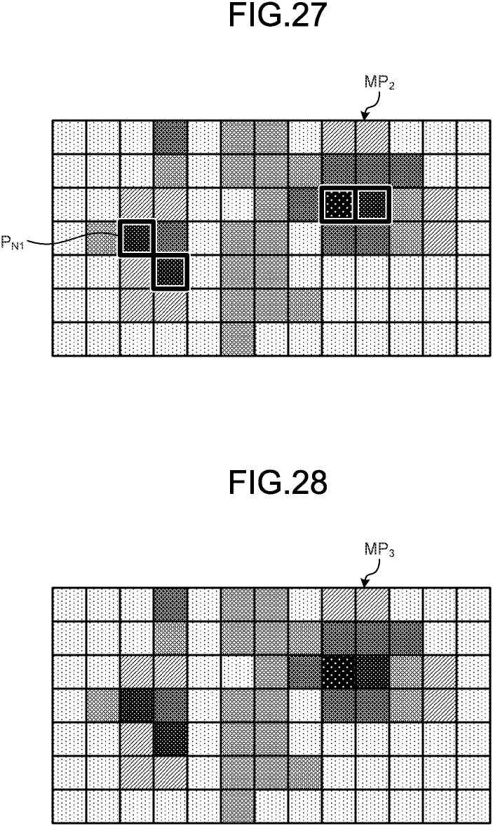

FIG. 28 is a diagram for describing identification of a variation grade in the second modification. The variation calculation unit 331 reads the feature data map MP₃ from the feature data map memory 323, and calculates the standard deviation of the feature data in the divided region from the feature data of each window data.

Thereafter, the variation calculation unit 331. refers to the association table in which the standard deviation and the variation grade are associated with each other, and identifies the variation grade corresponding to the standard deviation of the feature data for each divided region. FIG. 29 is a diagram for explaining identification of a variation grade. In the variation grade, as the standard deviation increases, the numerical value of the grade also increases. When the variation in the feature data in the divided region is large, the variation grade of the divided region is also large.

The subsequent processing is similar to that of the above-described embodiment.

In the second modification described above, the variation grade is calculated based on the standard deviation of the feature data for each of the divided regions in the feature data map, and the filter coefficient of the filtering process executed by the filter unit 342 is identified according to the variation grade. Therefore, in the second modification, as in the embodiment, it is easy to confirm the notable position of the tissue characterization in the ultrasound image having higher spatial resolution than the image based on the feature data. As a result, it is possible to display an image in which it is easy to search for a lesion having characteristics in tissue characterization without impairing spatial resolution.

### Other Modifications

Although the embodiments for carrying out the disclosure have been described so far, the disclosure should not be limited simply by the above-described embodiments. For example, in the ultrasound imaging apparatus, each unit may be configured by individual hardware, or all or some of the plurality of units may be configured by sharing an IC chip such as a CPU or a logic processor or other various types of hardware, and the operation may be realized by a software module.

Furthermore, in the present embodiment, the variation grade is identified based on the variation in the feature data devided region region in the scanning range, the in the devided region in the scanning range, variation map in which the variation grades of the respective divided regions are distributed in the scanning range is generated, and further, the relationship table in which the variation grade is associated with the information about the filter coefficient is used. With such a configuration and action, the variation itself of the feature data is indirectly associated with the filter coefficient to be applied to the position having the variation through the variation grade. However, a value, other than the variation grade, in which indirectly and uniquely connects the variation in the feature data and the filter coefficient to each other, may be used. Furthermore, the variation in the feature data and the filter coefficient may be directly and uniquely connected to each other.

Furthermore, in the present embodiment, an example is described in which the relationship table 1.1 which the variation grade and the information about the filter coefficient are associated with each other is output from the variation map generation unit 332 to the filter unit 342 via the characteristic selection data memory 333. However, the table may not be given or received such that the table is stored by the filter unit 342 or shared between the variation map generation unit 332 and the filter unit 342.

Furthermore, in the present embodiment, an example is described in which the low frequency band is enhanced as the setting of the filter coefficient. However, the overall filter passage ratio (input/output intensity ratio) may be increased in advance, and the passage ratio at the high frequency may be reduced. Also in this case, the low frequency component is enhanced, and the same effect as that of the embodiment can be obtained.

Furthermore, in the present embodiment, an example is described in which the “variation map” and the “relationship table in which variation grade is associated with filter coefficient information” are output as the characteristic selection data from the control unit 313 to the filter unit 342 via the characteristic selection data memory 333, but the control of the control unit 313 is not limited thereto. As the characteristic selection data, for example, curve data itself indicating the frequency characteristic indicating the passage ratio of the filter or other discrete data defining the frequency characteristic may be used.

In the present embodiment, the plurality of divided regions set in the scanning range Rₛ does not overlap each other. Alternatively, the adjacent divided regions may partially overlap each other. By partially overlapping, it is possible to generate an image without making the boundary of the divided region conspicuous. Here, overlapping of the divided regions means that there is a common Window.

Furthermore, in the present embodiment, a configuration may be employed in which a B-mode image generated without passing through the filter unit 342, that is, a B-mode image not subjected to the filtering process can be generated and displayed. At this time, a B-mode image subjected to the filtering process and a B-mode image not subjected to the filtering process can be displayed in parallel.

Note that, in the above-described embodiment, an example is described in which the feature data calculation unit 307 performs regression analysis to approximate the frequency spectrum with a linear expression (linear function) to acquire a regression line, and outputs a value of a preset type among the slope a₁, the intercept b₁, and the midband fit c₁ obtained from the regression line as the feature data. However, a value obtained by combining these types of values may be used as the feature data.

In addition, a value based on the slope a₁, the intercept b₁, and the midband fit c₁ may be used as the feature data. For example, it may be a nonlinear function such as an exponentiation, a weighted addition, or a combination of exponentiated values.

In addition, the attenuation correction process may be performed on the regression line obtained by the linear approximation, and the feature data may be calculated based on the regression line after the attenuation correction.

Furthermore, in the above-described embodiment, an example is described in which a regression line is generated by approximating the frequency spectrum by a linear expression (linear function) by performing regression analysis. However, the frequency spectrum may be approximated using a curve defined by a higher order polynomial (nonlinear function) of a second or higher order, or the frequency spectrum may be approximated by a finite power series. In addition, a curve defined by a polynomial of a trigonometric function or an exponential function may be used for approximation as the non-linear function.

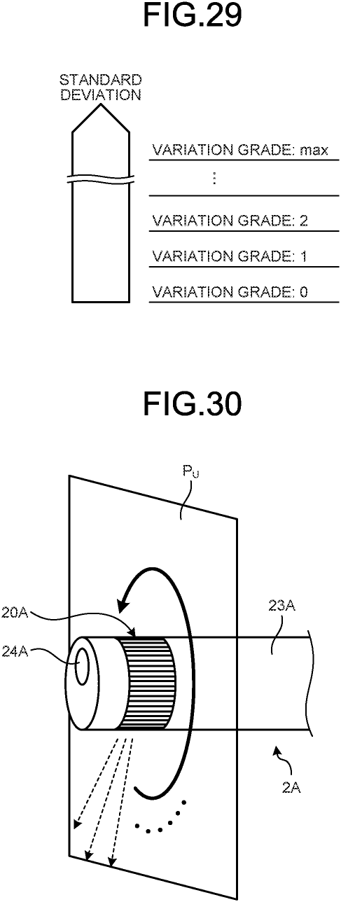

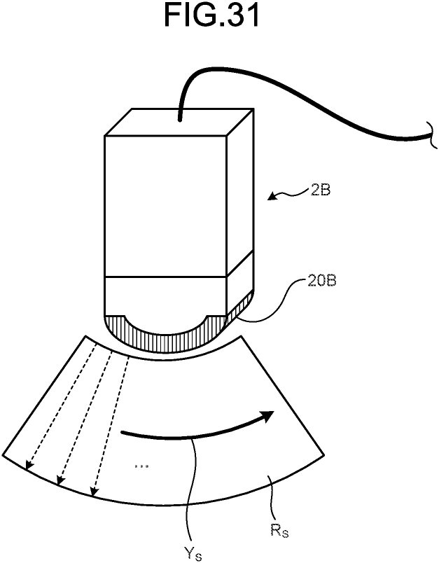

Furthermore, in the present embodiment, the convex type is described as an example of the ultrasound transducer, but the ultrasound transducer may be a linear type transducer or a radial type transducer. In a case where the ultrasound transducer is a linear transducer, the scan region has a rectangular shape (rectangle, square), and in a case where the ultrasound transducer is a radial transducer or a convex transducer, the scan region has a fan shape or an annular shape. FIG. 30 is a diagram (part 1) for explaining an example of the aspect of the ultrasound transducer. FIG. 30 illustrates a distal end configuration of an ultrasound endoscope as an ultrasound probe 2A. A distal end unit 23A of the ultrasound endoscope illustrated in FIG. 30 is provided with a radial ultrasound transducer 20A and an optical observation window 24A. The ultrasound transducer 20A transmits and receives ultrasound waves on a scanning face P_{U}. The ultrasound transducer 20A can rotate the transmission/reception direction of the ultrasound wave by 360°.

In the ultrasound transducer, piezoelectric elements may be two-dimensionally disposed. In addition, the ultrasound endoscope may cause the ultrasound transducer to perform mechanical scanning, or perform electronical scanning such that a plurality of elements is provided in an array as the ultrasound transducer, and elements related to transmission and reception are electronically switched or transmission and reception of respective elements are delayed.

Furthermore, in the present embodiment, the ultrasound probe is described using the ultrasound endoscope having the imaging optical system including the optical observation window, the optical lens, the imaging element, and the like, but the disclosure is not limited thereto, and an intraluminal ultrasound probe not having the imaging optical system may be applied. Specifically, a small-diameter ultrasound miniature probe may be applied. The ultrasound miniature probe is usually inserted into a biliary tract, a bile duct, a pancreatic duct, a trachea, a bronchus, a urethra, or a ureter, and is used for observing surrounding organs (pancreas, lung, prostate, bladder, lymph node, etc.).

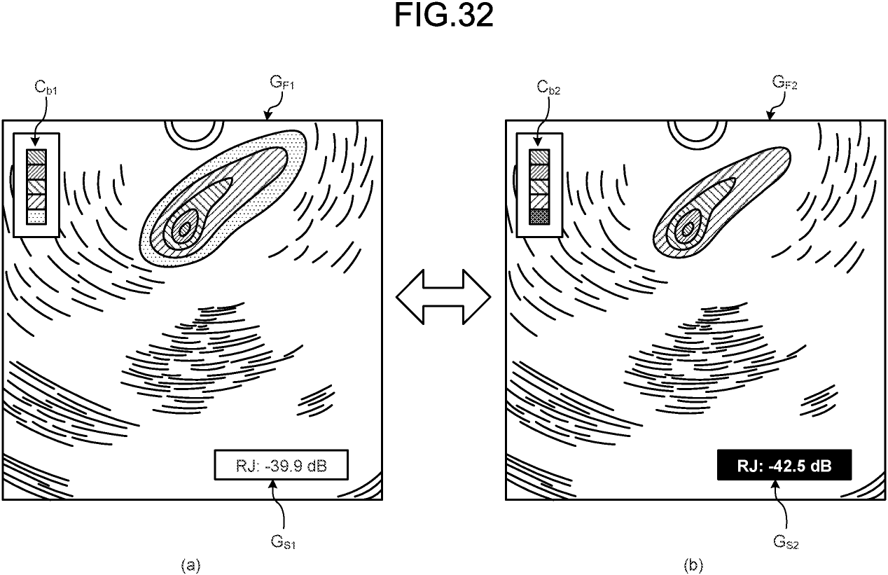

In addition, as the ultrasound probe, an external ultrasound probe that emits ultrasound at the body surface of the subject may be applied. The external ultrasound probe is usually used by being in direct contact with the body surface when abdominal organs (liver, gall bladder, bladder), breasts (particularly, mammary glands), and the thyroid gland are observed. FIG. 31 is a diagram (part 2) for explaining an example of the aspect of the ultrasound transducer. An external ultrasound probe 2B illustrated in FIG. 31 includes, for example, a convex ultrasound transducer 20B. The ultrasound probe 2B contacts, for example, a body surface of a subject and receives an ultrasound echo from a scattering body in the body.

In addition, the ultrasound imaging apparatus is not limited to a stationary type, but may be a portable or wearable apparatus.

Furthermore, in the above-described embodiment, the feature data image may be generated and displayed by providing visual information according to the feature data. For example, the control unit 313 generates the feature data image data in which the visual information related to the feature data generated by the interpolation process by the first interpolation unit 322 is allocated corresponding to each pixel of the image in the B-mode image data. FIG. 32 is a diagram for explaining a feature data image generated based on the feature data. For example, a feature data image G_{F1} illustrated in (a) of FIG. 32 is displayed on the display 4. The feature data image G_{F1} can be displayed side by side with the B-mode image and the feature data map described above.

In the feature data image G_{F1}, a color bar C_{b1} indicating the relationship between the feature data and the visual information and setting information G_{S1} such as a setting value are displayed on the feature data image. In FIG. 32, as the setting information G_{S1}, a setting value in the rejection function of eliminating (making transparent) coloring of the feature image is displayed. At this time, the selection color and the arrangement order of the visual information (color bar) corresponding to the value of the feature data can be set in any manner. Alternatively, the spatial filter may be applied before or after the coordinate transformation by the first coordinate transformation unit 321, or the necessity (ON/OFF) of the execution may be settable. Furthermore, the display mode of the setting value can be changed. For example, the image is changed to a feature data image G_{F2} illustrated in (b) of FIG. 32 having a mode in which cyan is used as the background color and a white numerical value is displayed thereon as the setting information G_{S2}. At this time, in a color bar C_{b2}, the visual information that is not displayed on the image due to the change of the setting value is displayed in black. In addition, when the change condition of the display mode of the setting value is stored as the user setting, the display may be returned to the standard display in which the background is white and the numerical value is displayed in black as illustrated in (a) of FIG. 32.

The disclosure may include various embodiments without departing from the technical idea described in the claims.

The ultrasound imaging apparatus, the operation method of the ultrasound imaging apparatus, and the operation program of the ultrasound imaging apparatus according to the disclosure described above are useful for visualizing a minute difference in tissue characterization as an ultrasound image.

According to the disclosure, it is possible to display an image in which it is easy to search for a lesion having characteristics in tissue characterization without impairing spatial resolution.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound observation system comprising a processor comprising hardware, the processor being configured to:
receive an echo signal based on ultrasound scanning of a scan region of a subject;
set first regions in the scan region, each one of the first regions including second regions;
calculate frequency spectra in the respective second regions based on an analysis of the echo signal;
calculate a plurality of pieces of feature data based on the frequency spectra;
calculate a statistical value of the plurality of pieces of feature data in the first regions;
set filters for the respective first regions based on the statistical value;
perform a filtering process with the filters on the echo signal to calculate a second echo signal; and
generate ultrasound image data based on an amplitude of the second echo signal,
frequency curves of the filters differing from each other depending on the statistical value.

2. The ultrasound observation system according to claim 1, wherein
the processor is configured to calculate, as the statistical value, a statistic, in the first regions, of the plurality of pieces of feature data associated with the second regions depending on a spatial distribution of the plurality of second regions included in the first regions.

3. The ultrasound observation system according to claim 1, wherein
the processor is configured to calculate, as the statistical value, a standard deviation, a variance, or an amount based on the standard deviation and the variance of the plurality of pieces of feature data in the first regions.

4. The ultrasound observation system according to claim 2, wherein
the processor is further configured to:
count the number of adjacent places where a difference between the plurality of pieces of feature data associated with the respective second regions included in the first regions and adjacent to each other is equal to or larger than a first threshold value, and
calculate a number density of the first regions based on the counted number as the statistical value.

5. The ultrasound observation system according to claim 1, wherein
the processor is further configured to:
count the number of second regions in which each of the plurality of pieces of feature data associated with the respective second regions is included in either a semi-bounded section or a bounded section defined by one or a plurality of second threshold values, and
calculate a number density of the first regions based on the counted number as the statistical value.

6. The ultrasound observation system according to claim 1, wherein
the filters are configured to:
perform weighting for each frequency based on the plurality of pieces of feature data.

7. The ultrasound observation system according to claim 6, wherein
the filters are configured to perform weighting in which a passage ratio of the echo signal at a low frequency is higher than a passage ratio of the echo signal at a high frequency.

8. The ultrasound observation system according to claim 1, wherein
the processor is configured to approximate the frequency spectra with a nonlinear function to calculate the plurality of pieces of feature data.

9. The ultrasound observation system according to claim 1, wherein
the processor is configured to approximate the frequency spectra with a linear function to calculate the plurality of pieces of feature data.

10. The ultrasound observation system according to claim 1, further comprising:
an ultrasound transducer configured to perform ultrasound scanning on the subject and transmit the echo signal to a receiver.

11. The ultrasound observation system according to claim 1, further comprising:
a display configured to display an ultrasound image based on the generated ultrasound image data.

12. An operation method of an ultrasound imaging apparatus, the method comprising:
receiving an echo signal based on ultrasound scanning of a scan region of a subject;
setting first regions in the scan region, each one of the first regions including second regions;
calculating frequency spectra in the respective second regions based on an analysis of the echo signal;
calculating a plurality of pieces of feature data based on the frequency spectra;
calculating a statistical value of the plurality of pieces of feature data in the first regions;
setting filters for the respective first regions based on the statistical value;
performing a filtering process with the filters on the echo signal to calculate a second echo signal; and
generating ultrasound image data based on an amplitude of the second echo signal,
frequency curves of the filters differing from each other depending on the statistical value.

13. A non-transitory computer-readable recording medium with an executable program stored thereon, the program causing an ultrasound imaging apparatus to execute:
receiving an echo signal based on ultrasound scanning of a scan region of a subject;
setting first regions in the scan region, each one of the first regions including second regions;
calculating frequency spectra in the respective second regions based on an analysis of the echo signal;
calculating a plurality of pieces of feature data based on the frequency spectra;
calculating a statistical value of the plurality of pieces of feature data in the first regions;
setting filters for the respective first regions based on the statistical value;
performing a filtering process with the filters on the echo signal to calculate a second echo signal; and
generating ultrasound image data based on an amplitude of the second echo signal,
frequency curves of the filters differing from each other depending on the statistical value.
